(19) United States
(12) Patent Application Publication
(10) Pub. No.: US 2025/0072954 A1
(43) Pub. Date: Mar. 6, 2025

(54) TREATMENT DEVICE AND CONTROL METHOD

(71) Applicant: Erbe Elektromedizin GmbH, Tuebingen (DE)

(72) Inventor: Thomas RIPPLINGER, Tuebingen (DE)

(21) Appl. No.: 18/810,029

(22) Filed: Aug. 20, 2024

(30) Foreign Application Priority Data
Aug. 31, 2023 (EP) 23194513.0

(51) Int. Cl. A61B 18/14; A61B 18/00; A61B 18/12

(52) U.S. Cl. CPC A61B 18/1442; A61B 18/1206; A61B 2018/00589; A61B 2018/00601; A61B 2018/00607; A61B 2018/00732; A61B 2018/00767; A61B 2018/1452

(57) ABSTRACT

Treatment devices having an instrument connected to a supplying apparatus. The instrument and the apparatus can control each other. An activation device is provided on the instrument or provided otherwise. The generator can be activated in a first or second operating type. The instrument comprises an alterable switch to guide the electrical power provided by the generator to a first or a second electrode pair or. The alterable switch is controlled by generator.

This application claims priority to European Patent Application No. 23194513.0, filed Aug. 31, 2023, the entirety of which is incorporated herein by reference.

The invention refers to a treatment device, which is particularly suitable for coagulation and cutting of biological tissue of a human or animal patient.

Treatment devices for tissue coagulation and/or cutting are known in different configurations.

EP 3 895 642 A1 describes such a treatment device consisting of a generator and an instrument, in which the generator is configured to produce an electrical voltage sufficient for coagulation of biological tissue. In the instrument a transformer is provided, which is connected to a cutting electrode and which is configured to create a voltage sufficient for cutting of tissue from the voltage configured for coagulation by transformation. An electrical switch, which can be activated by a push-button provided on the instrument, serves to activate the electrode arrangement. With this or an alternative second push-button an activation signal is created for the generator, in order to switch the latter on and off.

A treatment device having a generator and an instrument is known from EP 1 287 788 A1, wherein the instrument comprises a switch in order to alternatively connect a first or a second electrode pair with the generator. The control of the activation of the generator and the instrument is carried out by an activation device in form of a hand or foot switch. Accordingly, the power factor of the generator is switched.

Additional treatment devices are known from EP 1 545 360 A1, US 2010/0137854 A1, EP 1 599 146 B1 and EP 1 711 116 B1.

It is the object of the invention to provide an improved treatment device.

The treatment device according to the invention comprises a generator and an instrument supplied by the generator. The generator is configured to output an electrical voltage depending on a generator control signal, wherein the electrical voltage is suitable for a first treatment mode, for example tissue coagulation, or alternatively for a second treatment mode, for example tissue cutting. The generator is configured to be switchable between the two indicated treatment modes. As necessary, the generator can also be switchable between other and/or multiple treatment modes.

The generator is configured to be controlled by a generator control signal defining the treatment mode. In addition, the generator is configured to output an alterable switch control signal that controls an alterable switch preferably arranged in the instrument, in order to provide the voltage output by the generator and therefore also a current supplied by the generator to the electrode (or the respective electrode pair) suitable for the respectively selected treatment mode.

A signal carrier is used in the invention for transmission of the generator control signal as well as for transmission of the alterable switch control signal. The generator is configured to provide the signal carrier. The instrument is connected with the generator via a line and connected to the signal carrier so that the signal carrier is transmitted from the generator to the instrument. The signal carrier is preferably an alternating voltage in the sound frequency range between 20 Hz and 20 kHz, for example a square wave-shaped alternating voltage. Preferably, the frequency of the signal carrier is approximately 2 kHz. The signal carrier can be an alternating current signal that alternates with the indicated frequency, for example between a positive and a negative value. The positive and the negative value can have the same absolute value.

An activation device configured to produce the generator control signal can be assigned to the instrument. For this purpose, the activation device can be configured to apply different resistors with this alternating current signal for different desired operating modes. The resulting voltage is then the generator control signal, which is detected by a control device of the generator and is used for control thereof.

Alternatively, the signal carrier can also be an alternating voltage signal having a suitable frequency, for example 20 Hz to 20 kHz, for example 2 kHz, wherein in the instrument or in the activation device again different resistors, which can be activated via push-buttons, can be applied with the signal carrier. In this case the resulting alternating current is the generator control signal, wherein the control device of the generator is configured to control the generator accordingly.

The activation device can be arranged in the instrument or separate therefrom in an individual operating part.

According to the invention, the instrument of the treatment device comprises an alterable switch circuit that connects either the electrode provided for the first treatment mode or else the electrode provided for the second treatment mode with the line coming from the generator. Thereby for both electrodes one and the same counter-electrode can be provided that forms an electrode pair with the respective electrode of the selected treatment mode. The alterable switch can also be configured to switch an electrode of the first treatment mode as neutral electrode for the second treatment mode.

The alterable switch circuit is controlled by an alterable switch control signal according to the selected treatment mode. The generator is configured to create the alterable switch control signal and to impress it on the signal carrier. For example, the signal carrier can have a variable duty cycle, so that the average value of the signal carrier alternating between a positive and a negative value becomes either positive or negative. Thereby a first average value can be assigned to the first treatment mode and a second average value can be assigned to the second treatment mode. The alterable switch circuit can be configured to connect the first or second electrode with the line and thus the generator based on the detection of the different average values of the signal carrier respectively. The average value of the signal carrier is then the alterable switch signal.

The system according to the invention allows the control of the alterable switch provided in the instrument by the generator. The generator is in turn controlled by the activation device, which can be arranged in the instrument or a separate element, for example pedal.

In the invention the treatment current is supplied in controlled manner only to the electrode provided for the respective treatment. The alterable switch provided in the instrument is thereby controlled by the apparatus comprising the generator. The alterable switch is configured to connect only the electrode or those electrodes with the generator that are associated to the selected mode in which the apparatus operates.

Because the alterable switch controlled by the generator supplies current only to the activated electrode and no current flows via an electrode associated to a different mode, the generator voltage and/or the generator current can be detected particularly accurately and can be closed loop controlled sensitively. Based on the voltage and the current output by the generator as well as the phase shift between the two, the output power can be determined. In addition, the tissue resistance can be calculated and regulated as desired.

Quick alternating switching of the alterable switch and a respective guidance of the generator voltage, can activate both electrodes when supplied with current. For example, with a coagulation and cutting instrument a pre-sealing of a vessel or another tissue is made possible in this manner before the tissue cut is carried out in the context of cutting. If arteries or other vessels are held with a forceps-like instrument also comprising a cutting electrode, the coagulation electrodes can be applied with current first and the cut can be carried out in a delayed or slowed manner. This increases the treatment safety in that slipping of arteries, which are not completely sealed yet, out of the closed forceps-like instrument is avoided.

Another advantage of the treatment device according to the invention is that no transformer is required in the instrument, whereby the instrument can be provided particularly light and slim and with low technical efforts.

Preferably, the generator is a high frequency generator and configured to output an alternating voltage having a frequency between 100 kHz and 5 MHz. Preferably, the generator frequency is between 300 kHz and 600 kHz and can be varied with regard to its peak-to-peak voltage as well as, for example, in different current and/or power limitations. In addition, the generator can be configured to provide a modulated voltage, for example a voltage that is switched on and off, in order to obtain different desired crest factors.

The invention is particularly suitable for forceps-like instruments with immovable cutting electrode, wherein the first treatment mode can be a coagulation mode. For coagulation electrodes are provided on the two jaws in the forceps-like forked part. One of the two electrodes can be considered as neutral electrode, whereas the other electrode of the other jaw is the assigned coagulation electrode. The neutral electrode and the coagulation electrode form the electrode pair for tissue coagulation and/or tissue fusion.

The second treatment mode can be a cutting mode. In this mode the immovable cutting electrode is activated. In turn one of the two electrodes provided on the jaws can form the neutral electrode. It is also possible to configure the alterable switch circuit so that both electrodes provided on the jaws are electrically connected with one another in a cutting mode. The electrodes provided on the jaws are then the neutral electrodes in this mode.

Principally, the invention is also suitable for other instruments, for example instruments having a cutting and ablation function or the like.

With the concept according to the invention, it can be guaranteed that the generator control signal is set to start temporally after the alterable switch control signal and ends temporally before the alterable switch control signal. In doing so, it can be guaranteed that the generator outputs a voltage provided for one mode, if and only as long as the assigned electrode is connected with the generator.

In addition, the concept according to the invention allows to carry out automatic mode progresses owing to the control of the alterable switch provided in the instrument by the generator, during which subsequently different modes are used according to a provided program. For example, in a sealing and cutting instrument it can be guaranteed that the vessel or tissue sealing starts first and only thereafter the dissection (tissue cut) is carried out. In this case it is sufficient, if the activation device outputs a simple on/off signal as generator control signal. Then the mode progress can be controlled by the generator itself that also takes over the control of the instrument alterable switch.

Additional details of advantageous embodiments of the invention are subject of claims as well as the drawing and the associated description. The drawing shows:

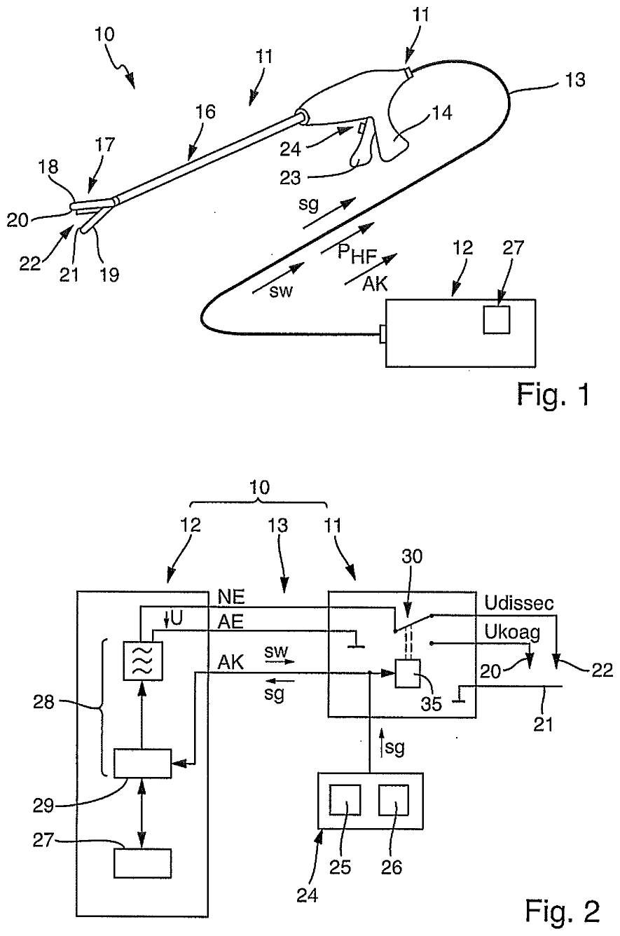

FIG. 1 an exemplary illustration of an instrument and an associated generator in schematic overview illustration,

FIG. 2 the treatment device according to the invention with generator and instrument in block diagram illustration,

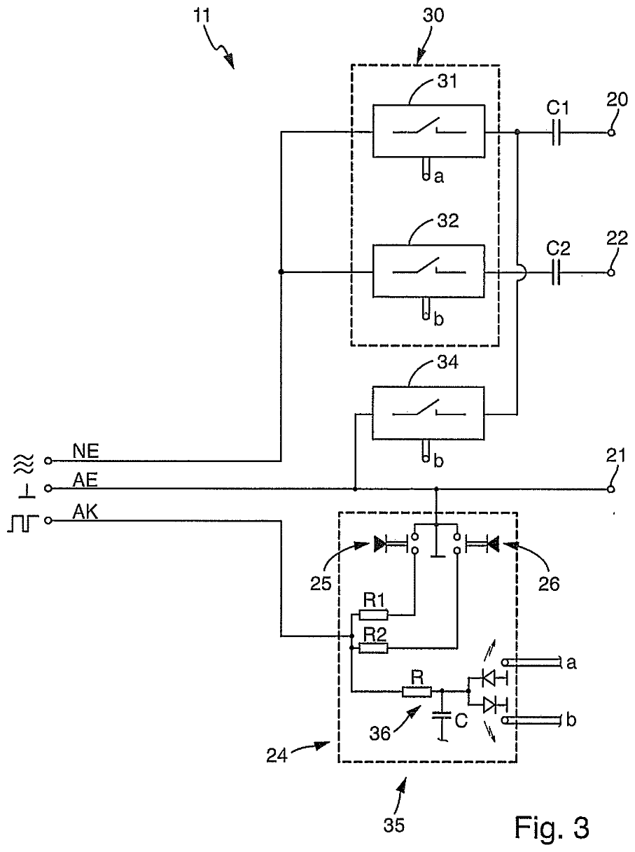

FIG. 3 the instrument being part of the treatment device according to the invention in block diagram illustration,

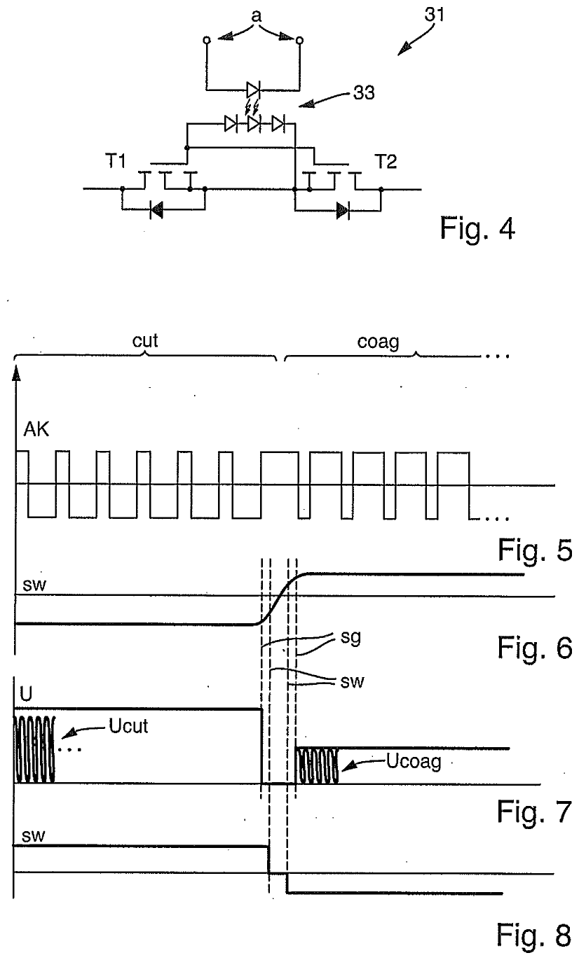

FIG. 4 an electronic switch used in the instrument according to FIG. 3,

FIG. 5 the signal carrier as time diagram,

FIG. 6 the alterable switch control signal in a temporal progress as schematic diagram,

FIG. 7 the high frequency voltage output by the generator as schematic diagram and

FIG. 8 the alterable switch control signal in form of a diagram resulting from the duty cycle of the signal carrier according to FIG. 5.

A treatment device 10 is illustrated in FIG. 1 comprising an instrument 11 as well as an apparatus 12 to which the instrument 11 is connected by a cable 13. The instrument 11 is illustrated in FIG. 1 as laparoscopic instrument by way of example. For this purpose, it comprises a handle 14 provided on a housing 15 from which a shank 16 extends in distal direction at the end of which a forceps-like tool 17 is held. The tool 17 serves for tissue coagulation and tissue cutting, i.e. for example, for sealing vessels, for example blood vessels, and the cutting thereof.

The tool 17 comprises coagulation electrodes 20, 21 at its jaws 18, 19, between which tissue can be grabbed and can be held in compressed manner. Due to current flow between the coagulation electrodes 20, 21, the tissue is heated and coagulated. For example, blood vessels and other hollow structures can be fused thereby and thus sealed.

At least one of the jaws 18, 19 is additionally provided with a cutting electrode 22. This cutting electrode serves to cut through tissue. It operates typically with a higher voltage than the coagulation electrodes and with higher local current concentration.

The instrument 11 is in this form only indicated for exemplary illustration. It can also be configured as instrument for the open surgical use or the endoscopic use. Instead of the coagulation electrodes 20, 21 and the cutting electrode 22 also electrodes for other treatment purposes can be provided, for example ablation electrodes or the like, that can be activated at different points in time and for this purpose connected with the generator by a switching device.

For opening and closing the jaws 18, 19 at least one operating element 23 is arranged on the housing 14, for example in the form of a hand lever. In addition, an activation device 24 having one or more switches or push-buttons 25, 26 (see FIG. 2) can be provided for activation of the electrodes 20, 21, 22 (see FIG. 1). The activation device 24 can be part of the instrument 11, as illustrated in FIG. 1. Alternatively, the activation device 24 can also be arranged in a separate housing and can be configured, for example, as hand or foot switch.

In addition, apparatus 12 can have an operating unit 27 by which the operation of instrument 11 can be influenced. The operating unit 27 can be configured to set the operating parameters for the treatment modes coag, cut or to carry out other adjustments.

The treatment device 10 is illustrated individually and in abstract manner in FIG. 2. The apparatus 12 comprises a generator 28 that is configured to output a high frequency voltage U suitable for carrying out different treatments on biological tissue. The voltage U is preferably a high frequency voltage, the peak value of which can be varied in a range of approximately 100 V to multiple 100 V or also more. In addition, generator 28 is preferably configured to modulate this high frequency voltage. For example, voltage U can be output as continuous voltage or also as on/off-switched voltage, wherein the pulse/pause-ratio can be variable according to the desired mode of the treatment. For example, the generator 28 can be configured to output the voltage Ucoag for a first treatment mode coag, for example a coagulation mode, in which the voltage shall be effective between the two electrodes 20, 21. In addition, generator 28 can be configured to output a voltage Ucut for a second treatment mode cut, for example a cutting mode, which is sufficient for tissue cut and shall be effective between the electrodes 22, 21.

The control of the generator is carried out according to a generator control signal sg. A control device 29 can be provided that is configured to evaluate the generator control signal sg and to use it for control of generator 28. The control device 29 can be part of the apparatus 12 and can also be connected with operating unit 27.

The cable 13 comprises at least three lines NE, AE and AK leading to the instrument 11. While line AE supplies neutral potential, generator 28 outputs the treatment voltage and the treatment current to the instrument 11 via line NE.

Referring to FIG. 3, instrument 11 comprises an electrical alterable switch 30 by which the treatment voltage and the respective treatment current coming from line NE is selectively guided to the electrode 20 provided for coagulation or the electrode 22 provided for cutting. The alterable switch 30 is in so far a switching device switched by an alterable switch control signal sw by control device 29 being part of the generator 28. The alterable switch control signal sw is transmitted via line AK. Similarly, the generator control signal sg is transmitted via line AK.

The alterable switch comprises two electronic switches 31, 32 that can be configured according to the example of FIG. 4. FIG. 4 exemplarily illustrates for this purpose switch 31 with two field effect transistors T1, T2 orientated with opposite polarity and switched in series, the gate electrodes of which can be controlled via an optocoupler 33 in potential-free manner. The optocoupler 33 receives signal a at its input, which is provided by the activation device 24 (FIG. 3). The switch 31 can be connected with the electrode 22 via a coupling capacitor C1. Similarly the second switch 32 can be connected with electrode 22 via a coupling capacitor C2.

As an option, as depicted in FIG. 3, a third switch 34 connecting the capacitor side output of switch 31 with the neutral line AE upon receiving the control signal b can in addition be part of the alterable switch 30. This switch 34 can be used to keep electrode 20 free from voltage, while electrode 22 serving for cutting is applied with current. In this case, both electrodes 20, 21 together form the neutral electrode for the cutting voltage originating from cutting electrode 22.

Electrical switches 25, 26, for example in form of push-buttons, provided in or on the activation device 24, serve for switching on and toggling generator 28. As apparent from FIG. 3, these switches 25, 26 are switched in series with resistors R1, R2 between neutral potential on the line AE and the line AK. The line AK guides a signal carrier. For communication of the activation device 24 with control device 29 as well as for communication of control device 29 with alterable switch 30, the generator control signal sg and the alterable switch control signal sw can be impressed on the signal carrier.

The resistors R1 and R2 can have different values in order to influence the signal carrier on line AK for creation of the generator control signal sg in different ways. For example, signal carrier on line AK is a square wave alternating current symmetrically to zero having a frequency of, for example, 2 kHz with which it alternates between two current values of +25 mA and −25 mA. According to the value of the resistor R1 or R2 activated by actuation of switch 25 or 26, control device 29 detects the resulting voltage as generator control signal sg. Accordingly, it activates the generator 28 either in the first or in the second operating mode, i.e. for example, either in the coagulation mode coag or in the cutting mode cut, whereupon generator 28 outputs the required voltage Ucoag or Ucut (FIG. 7).

The signal carrier on line AK can also be a voltage signal alternating between a positive and a negative voltage value with a defined frequency, for example 2 kHz. The control device 29 then detects the resulting current as generator control signal sg and makes generator 28 to operate in the signaled operating type accordingly.

The control device 29 is in addition configured to produce an alterable switch control signal sw for controlling the alterable switch 30 and to output it via line AK after detection of a generator control signal sg. The alterable switch control signal sw is detected in the instrument 11 by a detector device 35, which can be configured as average value detector 36 (FIG. 3).

The control device 29 is configured to encode the alterable switch control signal sw in the duty cycle of the signal carrier. This is exemplarily illustrated in FIG. 5. The duty cycle here means the ratio of the duration of a positive voltage or a positive current compared to the temporal duration of the negative voltage or the negative current within one wave train illustrated in FIG. 5. The duty cycle in the left part of the diagram of FIG. 5 characterized with “cut” is less while it is high during the right phase characterized with the term “coag”. For detection of these ratios serves the average value detector 36 that can be configured in form of an RC low pass, for example. The output of the low pass can be connected with light emitting diodes, for example of optocouplers, forming the inputs of the respective electronic switches 31, 32 (and as appropriate 34). These light emitting diodes are schematically illustrated in the activation device 24 in FIG. 3. However, they can also be arranged in the switches 31, 32, 34, as obvious from FIG. 4. In addition, between the light emitting diodes or other inputs of switches 30, 32, 34, and the average value detector 36, switching or amplification components can be arranged, the preferably high ohmic inputs of which stress the average value detector 36 and thus the voltage on capacitor C little or not at all.

The treatment device 10 described so far operates as follows:

After start-up instrument 11 is connected to apparatus 12, as illustrated in FIGS. 1 and 2. If now, for example, biological tissue (e.g. a blood vessel or another vessel) shall be sealed and cut through, it is gripped by jaws 18, 19, which are closed by the operating device 23 and compress the tissue. The activation device 24, particularly the activation of switch 25, activates the generator 28. This is carried out in that switch 25 is closed, whereby current flows through resistor R1 and via line AK. This is detected by control device 29 as generator control signal sg.

The generator control device 29 interprets the generator control signal sg as command for taking the coagulation mode and impresses the modulation illustrated on the right side in FIG. 5 on the signal carrier on line AK. The duty ratio is high, whereby after low pass filtering the positive alterable switch control signal sw, according to the right side in FIG. 6, is detected and provided by average value detector 26 as control signal a to switch 31. The switch 31 closes, i.e. the alterable switch 30 connects the electrode 20 with line NE and thus with the high frequency generating part of generator 28. The other switches 32, 34 remain open (non-conductive). In doing so, the coagulation voltage Ucoag is applied to electrode 20, whereby the coagulation current can flow from electrode 20 to electrode 21.

If it shall be changed to the cutting mode, the user operates the other switch 26. Now the second resistor R2 is switched between the neutral potential of line AE and the signal carrier on line AK. This is interpreted by control device 29 as generator control signal sg for the cutting mode. Accordingly, it changes the modulation of the signal carrier (line AK) according to the left side in FIG. 5.

According to the generator control signal sg, the generator voltage U has now, as shown in FIG. 7, a different value Ucut (left) compared to the coagulation mode Ucoag (right). The average value of the signal carrier is now negative, which is the reason for detector 35 to open switch 31 and to close switches 32, 34. If this has happened, the high frequency voltage U is now applied to output 22 as cutting voltage Ucut having a respective higher amplitude, as illustrated in FIG. 7 on the left side. The cutting voltage Ucut is guided via alterable switch 30 to electrode 22 while switch 34 is closed. The two electrodes 20, 21 are therefore connected in parallel and together form the neutral electrode for the cutting electrode 22.

As apparent from FIG. 8, the changeover switching of the alterable switch and the switching on and off of the high voltage part of generator 28 is carried out with temporal offset. The closing of switches 31, 32, 34 is carried out always prior to the activation of the high voltage part of generator 28. Vice versa the generator 28 is always switched off before the respective switch of the alterable switch 30 is opened. This is apparent from the temporal sequence of the generator control signal sg and the alterable switch control signal sw illustrated in FIGS. 6 and 7.

The described changeover switching process can be carried out one time or multiple times sequentially also in quick sequence in order to achieve a virtual concurrent cutting and coagulating, for example. During the first operating mode, for example coagulation, as well as during the second operating mode, for example cutting, it is possible to monitor a current and voltage of the generator or the power output by the generator or the tissue condition accurately and to control the generator accordingly. In addition, the current and the voltage are individually adjustable for each operating mode coag, cut on apparatus 12.

Besides the manual changeover between different operating modes described above, it is also possible to store and retrieve different treatment progresses in the generator 28. In this case, generator 28 controls the alterable switch 30 according to the predefined treatment progress after a start signal has been output from the activation device 24 and outputs the respective voltages and currents to the respectively activated electrodes 20 or 22 depending on the alterable switch position.

The treatment device 10 according to the invention comprises an instrument 11 connected to supplying apparatus 12. The instrument 11 and the apparatus 12 can control each other. By means of an activation device 24 provided on the instrument 11 or provided otherwise, the generator can be activated in a first or second operating type. The instrument 11 comprises an alterable switch 30 to guide the electrical power provided by the generator to a first or a second electrode pair 20/21 or 22/21. The alterable switch 30 is controlled by generator 12. This offers the possibility to store treatment progresses in the generator and to carry them out with the instrument 11 without requiring the user to change operating types.

1. A treatment device for coagulation and cutting of biological tissue of a human or animal patient, comprising:
a generator configured to output a first value (Ucoag) for a first treatment mode (coag) and a second value (Ucut) for a second treatment mode (cut) as well as to output an alterable switch control signal (sw) based on a first condition or a second condition being different from the first condition;
an instrument having at least two electrodes connected to the generator via an electrical line;
an electrical alterable switch arranged between the electrodes and the line; and
an activation device that is configured to produce a generator control signal (sg) to activate the generator.

2. The Treatment device according to claim 1, wherein the generator is a high frequency generator configured to output a voltage having a frequency between 100 kHz and 5 MHz.

3. The treatment device according to claim 1, wherein the first treatment mode (coag) is a mode for tissue coagulation.

4. The treatment device according to claim 1, wherein the second mode (cut) is a mode for tissue cutting.

5. The treatment device according to claim 1, wherein the first and second values represent the peak-to-peak voltage measured from the positive peak to the negative peak, and wherein the first value (Ucoag) is is smaller than the second value (Ucut).

6. The treatment device according to claim 1, wherein the generator comprises a control device configured to send a signal carrier to the activation device via a line (AK).

7. The treatment device according to claim 6, wherein the activation device is configured to generate the generator control signal (sg) from the signal carrier depending on a user input.

8. The treatment device according to claim 6, wherein the activation device comprises at least two switches that are connected with different electrical resistors (R1, R2).

9. The treatment device according to claim 6, wherein the activation device is arranged in the instrument.

10. The treatment device according to claim 6, wherein the signal carrier comprises the alterable switch control signal (sw).

11. The treatment device according to claim 6, wherein the signal carrier is an alternating signal having a variable duty ratio.

12. The treatment device according to claim 6, wherein the alterable switch control signal (sw) is defined by the duty ratio of the signal carrier.

13. The treatment device according to claim 6, further comprising an average value detector configured to extract the alterable switch control signal (sw).

14. The treatment device according to claim 6, wherein the generator control signal (sg) is defined to temporally start after the alterable switch control signal (sw) and to temporally end before the alterable switch control signal (sw).

15. A method for controlling a treatment device, comprising:
Supplying a signal carrier output from a generator to an activation device;
supplying a generator control signal (sg) to the generator, the control signal (sg) corresponding to a desired treatment mode (coag, cut);
supplying an alterable switch control signal (sw) from the generator,
the alterable switch control signal (sw) determined in the instrument from the signal carrier and supplied to the alterable switch,
activating the electrode suitable for the desired treatment mode (coag, cut) with the line (NE).
